(19) United States
(12) Patent Application Publication
(10) Pub. No.: US 2013/0071429 A1
(43) Pub. Date: Mar. 21, 2013

(54) BUNYAVIRUS VACCINE

(71) Applicants: Benjamin Brennan, St Andrews (GB); Richard Michael Elliott, St Andrews (GB)

(72) Inventors: Benjamin Brennan, St Andrews (GB); Richard Michael Elliott, St Andrews (GB)

(73) Assignee: UNIVERSITY COURT OF THE UNIVERSITY OF ST ANDREWS, St Andrews, Fife (GB)

(21) Appl. No.: 13/576,672

(22) PCT Filed: Jan. 28, 2011
(86) PCT No.: PCT/GB2011/000119

§ 371 Date: Oct. 5, 2012

(30) Foreign Application Priority Data
Feb. 3, 2010 (GB) 1001726.7

(51) Int. Cl. A61K 39/12; A61P 37/04; C12N 7/01; C12N 1/21; C12N 15/63; C12N 5/10; C12N 1/15; A61P 31/14; C12N 7/02

(52) U.S. Cl. 424/204.1; 435/235.1; 435/239; 435/320.1; 435/419; 435/348; 435/325; 435/254.11; 435/252.3; 435/364; 435/352

(57) ABSTRACT

The present invention provides attenuated viruses for use as vaccines and for the treatment and/or prevention of viral diseases and/or infections.

### FIELD OF THE INVENTION

The present invention provides attenuated viruses for use as vaccines and for the treatment and/or prevention of viral diseases and/or infections.

### BACKGROUND OF THE INVENTION

Rift Valley fever virus (RVFV) is a mosquito-borne pathogen of both livestock and humans. The virus was originally isolated in Kenya and caused major epidemics primarily in sub-Saharan Africa. However, the virus spread to Egypt and west Africa in 1977/78 and then to the Arabian peninsula in 2000.

In ruminants, RVFV disease is characterised by foetal deformities, abortion and high rates of mortality due to hepatitis among young animals that can approach 100%. In man, infection generally results in self-limiting febrile illness, but the disease can progress to hemorrhagic fever, encephalitis or retinal vasculitis. During an outbreak in Saudi Arabia an estimated 2000 human cases were reported along with 245 deaths (Shoemaker et al., 2002). The more recent outbreak of RVFV in 2000 in Sudan (World Health Organisation: Global Alert and Response: http://www.who.int/csr/don/2008_{—}01_{—}22/en/index.html) resulted in 698 human cases with 222 deaths, a case fatality rate of 31.8% (W.H.O, 2008). RVFV can be transmitted by a wide range of dipteran hosts, and remains a potential worldwide human, animal and economical threat (Elliott, 2009).

RVFV is a member of the Phlebovirus genus in the Bunyaviridae family. It contains a tripartite RNA genome comprising two negative-sense, and one ambisense, segments that code for seven viral proteins. The large (L) segment (approx 6.4 kb in length) encodes the viral RNA-dependent RNA polymerase. The medium (M) segment (approx. 3.8 kb) codes for four proteins in a single open reading frame (ORF): the envelope glycoproteins Gn and Gc, and two non-structural proteins designated NSm1 and NSm2, depending on which of the five potential methionine start codons initiates translation (Won et al., 2006, Gerrard et al., 2007a). The small (S) segment (approx. 1.6 kb) encodes the nucleocapsid protein (N) in a negative-sense orientation whereas the non-structural protein NSs, is encoded in the virus genomic sense RNA. It has been demonstrated that the non-structural proteins, NSm and NSs, function as virulence factors and determinants of mammalian host pathogenesis (Billecocq et al., 2004, Bird et al., 2007a, Won et al., 2007, Bird et al., 2008, Habjan et al., 2009, Ikegami et al., 2009b, Ikegami et al., 2009a).

Due to the potential for devastating outbreaks of RVFV amongst cattle, with the associated economic losses, and for fatal disease in humans, a great deal of effort has gone into RVFV vaccine development. Many different avenues are being explored as potential vaccine candidates, such as live-attenuated viruses, formalin-inactivated vaccines, heterologous viral vectors expressing RVFV proteins, RVF virus-like particles or DNA vaccines (Bouloy and Flick, 2009). Currently there are no vaccines licensed for use in humans, and the licensed animal vaccines either elicit poor immunogenicity or have severe side effects, similar to that of RVFV-induced disease (Smithburn, 1949, Vialat et al., 1997, Bouloy and Flick, 2009, Kamal, 2009). Promising results have been obtained with live-attenuated viruses, that express a deleted form of NSs, or have had NSs (and in some cases NSm) deleted. However, these live attenuated viruses all have the potential for reversion to virulence via genetic reassortment with wild-type virus—as might happen during a rampant RVFV epidemic.

An object of the present invention is to develop a Phlebovirus for use as a vaccine, exhibiting attenuated growth in mammalian cell culture, competency to synthesise all viral proteins necessary for the production of infectious virus particle, an impaired ability to inhibit host protein synthesis and a restricted ability to undergo genetic reassortment during mixed infection.

### SUMMARY OF THE INVENTION

The present invention relates modified viruses for raising immune responses in animals. In particular, the invention provides viruses of the Bunyavirus family (the Bunyaviridae) modified so as to be genetically distinct from corresponding wild-type (or un-modified) Bunyavirus family species. That is to say, when compared to the genome of a corresponding wild-type species, the genomes of the modified viruses provided by this invention comprise one or more nucleic acid alterations/modifications.

The modified viruses described herein may, for example, find application as vaccines for the treatment and/or prevention of diseases caused or contributed to, by members of the Bunyavirus family such as, for example, those belonging to the Phlebovirus genus.

As such, according to a first aspect, this invention provides a modified virus of the Bunyavirus family, said modified virus comprising a bi-segmented genome.

Viruses of the Bunyavirus family comprise a segmented RNA genome comprising negative sense RNA. In most cases the genome comprises three negative sense RNA segments. In some cases, at least one of the RNA segments has an ambisense arrangement. More specifically, the Bunyavirus family (otherwise known as the Bunyaviridae) comprises a number of different viral genera including, for example, the Hantavirus genus, the Nairovirus genus, the Tospovirus genus, the Orthobunyavirus genus and the Phlebovirus genus. As such, the invention described here is applicable to viral species belonging to each of the genera within the Bunyavirus family.

In one embodiment, the modified virus of the Bunyavirus family is a modified virus of the Phlebovirus genus. The Phlebovirus genus comprises a number of species responsible for severe disease, especially mammalian (particularly ungulate, ruminant, for example ovine or bovine) infections and diseases. As such, the term “Phlebovirus” encompasses all such species including, for example those known as Alenquer virus, Candiru virus, Chagres virus, Naples virus, Punta Toro virus, Rift Valley fever, Sicilian virus and Toscana virus. Other viruses (for example members of the Bunyavirus family or Phelbovirus genus) for modification in accordance with the methods and protovcols described herein may include, for example, those described by Nichol et al., 2005 (Vitus taxonomy; Eighth report of the International Committee on Taxonomy of Viruses).

In one embodiment, the invention provides a modified Rift Valley fever virus (RVFV).

It should be understood that hereinafter, use of the general term “Phlebovirus” encompasses all of the different Phlebovirus species of the Phlebovirus genus. Furthermore, while the invention is described with particular reference to the Phlebovirus genus and in particular RVFV, the methods, protocols and techniques described herein are applicable to all members of the Bunyavirus family (Bunyaviridae) including those described above. More generally, the invention may be considered applicable to viruses having segmented negative sense RNA genomes as exemplified by the Bunyaviridae.

As stated, modified viruses, for example modified Phleboviruses, provided by this invention are genetically distinct from wild-type Phlebovirus which harbour a tripartite genome comprising L (large), M (medium) and S (small) RNA segments. In wild-type Phlebovirus, these three RNA segments encode seven proteins. Specifically, the L segment encodes a RNA-dependent RNA polymerase, the M segment encodes two glycoproteins (Gn and Gc) and two non-structural proteins designated NSm1 and NSm2 in a single open reading frame. The S segment is an ambisense segment encoding a nucleocapsid protein (N) and another non-structural protein termed—NSs.

In contrast, the genome of the modified virus provided by this invention may comprise, consist or consist essentially of, a large (L) segment and a modified small (mS) segment. As such, the genome of the modified virus lacks an M segment

In one embodiment, the L segment of the modified virus described herein is a wild-type L segment encoding a viral RNA-dependent RNA polymerase. In a further embodiment, the L segment of the modified viral genome, may include one or more modifications. For example, the nucleic acid of the L segment may be modified to include one or more nucleic acid insertions (additions), deletions and/or inversions (see definitions below). In one embodiment, the nucleic acid of the L segment may be modified to include one or more nucleic acid sequences encoding amino acid, peptide, polypeptide or protein moieties which may be used as tags of labels. For example, the amino acid, peptide, polypeptide or protein tag or label may define one or more epitopes. One of skill will appreciate that by modifying L genome segments to include some form or detectable tag or label, it is possible to screen viral isolates and determine their genetic origin.

The mS segment may be modified in such a way so as to disrupt the sequence encoding the NSs protein (referred to hereinafter as the NSs sequence). It should be understood that disruption of the NSs sequence may prevent, ablate or reduce expression of the NSs protein by the modified virus. One of skill in this field will understand that disrupted NSs sequences may comprise one or more nucleic acid additions (or insertions), deletions and/or inversions.

In one embodiment, all or part of the NSs sequence may be deleted in order to prevent, reduce or ablate NSs expression. In other embodiments, one or more nucleic acid sequences (including one or more single or polynucleotide sequences) may be inserted (added) to the NSs sequence—disruptions of this type can shift reading frames leading to reduced or ablated functional NSs expression. In other embodiments, disruptions to the NSs sequence may include the inversion of two or more nucleotides of the NSs sequence.

In view of the above, the modified Phlebovirus may comprise a bi-segmented genome comprising, consisting or consisting essentially of a wild-type L segment and a modified S segment comprising a disrupted NSs sequence. In one embodiment, the modified Phlebovirus described herein lacks a NSs sequence.

In other embodiments, the modified virus provided by this invention comprises an mS segment modified such that the NSs sequence is replaced with one or more heterologous sequence(s) or one or more sequence(s) normally encoded by another Bunyavirus family member or genome segment—for example, one or more gene sequences from the L and/or M segments.

Where, for example, the modified virus is a modified Phlebovirus, it should be understood that the term “heterologous” nucleic acid sequence encompasses sequences not normally encoded by the Phlebovirus S genome segment as well as sequences not normally encoded by the Phlebovirus genome. For example, the heterologous nucleic acid sequence may encode proteins normally encoded by the L and/or M segments of the Phlebovirus genome. Additionally or alternatively, the heterologous nucleic acid sequences may encode proteins not normally encoded by the viral genome such as, for example, marker proteins such as GFP, eGFP (enhanced GFP), or hREN (humanised Renilla Luciferase

In one embodiment, the NSs sequence of the modified viruses described herein is replaced with a sequence encoding the Phlebovirus glycoproteins Gn and Gc. As such, the mS segment may otherwise be referred to as a chimeric segment having an ambisense arrangement and/or encoding the Phlebovirus N protein and the Phlebovirus glycoproteins, Gn and Gc. In one embodiment, the sequence encoding the Gn and Gc glycoproteins is obtained or derived from the M segment. Where the modified virus is a modified RVFV, the sequence encoding the glycoproteins, Gn and Ge is derived from the RVFV M segment.

In one embodiment, the invention provides a modified RVFV, said modified RVFV comprising a bi-segmented genome consisting essentially of a wild-type RVFV L segment and a modified RVFV S segment, said modified RVFV S segment comprising a disrupted NSs sequence and encoding the RVFV N protein and the RVFV glycoproteins, Gn and Gc. In one embodiment, the RVFV NSs sequence is deleted and replaced with a sequence encoding the RVFV Ge and Gn glycoproteins.

The inventors have observed that modified virus produced according to this invention exhibits attenuated growth in mammalian cell culture. Furthermore, while the modified virus is able to synthesise all proteins necessary for the production of infectious virus particles, it is unable to inhibit host protein synthesis.

Furthermore, the inventors hypothesise that in contrast to prior art modified Bunyavirus family members comprising tripartite genomes from which nucleic acid sequences have been deleted, the bi-segmented modified virus described herein may not revert to a virulent form when mixed with wild-type or three-segmented Phlebovirus. Modified viruses, for example modified Phleboviruses which exhibit a restricted or ablated ability to revert to virulence following reassortment with wild-type strains, are particularly useful as vaccines as they are much less likely revert to virulence during a disease epidemic.

In view of the above, the modified virus (for example modified Bunyavirus family member or modified Phlebovirus), provided by the first aspect of this invention, may otherwise be referred to as an attenuated Bunyavirus family member or attenuated Phlebovirus. Where the invention concerns a modified RVFV, the modified virus may be referred to as an attenuated RVFV.

In a second aspect, the present invention provides a modified virus according to the first aspect of this invention, for raising an immune response.

In one embodiment, the second aspect of this invention provides vaccines or vaccine compositions comprising the modified virus described herein for raising immune responses. Vaccines of this type may be known as attenuated vaccines.

Vaccines or vaccine compositions provided by the second aspect of this invention may be used prophylactically to prevent infection by Bunyavirus family members, for example Phlebovirus genus viruses. In some cases the vaccines provided by this invention may be used to reduce infection or the symptoms thereof. In yet further embodiments, the vaccine or vaccine compositions provided by this invention may be used to reduce colonisation of a host by a Bunyavirus. Subjects exposed to the vaccines provided by this invention, may produce antibodies which bind to (or which exhibit affinity or specificity for) the Bunyavirus or antigens thereof—such antibodies may otherwise be referred to as “protective” antibodies. Accordingly, the vaccines or vaccine compositions provided by this invention may be used to induce, elicit or raise a protective immune response or antibodies in an animal.

The Bunyavirus family, and in particular the Phlebovirus genus, comprises a number of species responsible for severe animal (particularly human and ruminant, for example bovine or ovine) infections and disease and the vaccines and vaccine compositions described herein may be used to treat and/or prevent diseases caused or contributed to, by such viral species. It should be understood that the term “animal” encompasses mammalian species including, for example, ungulate or ruminant (for example ovine or bovine) species as well as primates including, for example, humans. Rift Valley fever (RVF) is caused by RVFV and is a vector (mosquito) borne disease affecting both livestock (ruminants) and humans and as such, the vaccines and vaccine compositions provide by this invention may be use to treat and/or prevent RVF.

The modified Phleboviruses provided by this invention may be formulated as vaccine compositions (preferably sterile vaccine compositions) comprising a pharmaceutically acceptable carrier or excipient. Such carriers or excipients are well known to one of skill in the art and may include, for example, water, saline, phosphate buffered saline, dextrose, glycerol, ethanol, ion exchangers, alumina, aluminium stearate, lecithin, serum proteins, such as serum albumin, buffer substances such as phosphates, glycine, sorbic acid, potassium sorbate, partial glyceride mixtures of saturated vegetable fatty acids, water salts or electrolytes, such as protamine sulphate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polypropylene-block polymers, polyethylene glycol and wool fat and the like, or combinations thereof. The vaccine compositions provided by this invention may be formulated for oral, parenteral (including subcutaneous, intradermal, intramuscular and intravenous), and mucosal (for example nasal) administration. By way of example, vaccine compositions for parenteral administration include sterile solutions or suspensions of modified virus in aqueous or oleaginous vehicles. Injectable preparations may be adapted for bolus injection or continuous infusion. Such preparations are conveniently presented in unit dose or multi-dose containers, which are sealed after introduction of the formulation until required for use.

It should be understood that in addition to the aforementioned carrier ingredients the vaccine compositions described above may comprise one or more additional carrier ingredients such as diluents, buffers, flavouring agents, binders, surface active agents, thickeners, lubricants, preservatives (including anti-oxidants) and the like, and substances included for the purpose of rendering the composition isotonic with the blood of the intended recipient.

Pharmaceutically acceptable carriers are well known to those skilled in the art and include, but are not limited to, 0.1 M and preferably 0.05 M phosphate buffer or 0.8% saline. Additionally, pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

One of skill will appreciate that the dose and/or administration regime used to vaccinate any given animal or animal population may vary depending on, for example, the modified virus to be used as a vaccine and the host to be immunised. Generally speaking the dose should be sufficient to induce at least a temporary immunity to a Phlebovirus (for example RVFV) infection. One of skill will appreciate that by administering a number of doses over a period of time, it may be possible to increase the length of time a vaccinated host retains immunity to Phlebovirus infection/disease. Typically, the dose is sufficient to raise, elicit or induce an immune response/protective antibodies.

Modified virus for use in the present invention may be obtained using recombinant techniques. One of skill will appreciate that, for example, PCR based techniques can be used to excise specific nucleotide sequences from larger nucleic acid sequences. As such, PCR based excision techniques may be used to, for example, excise all or part of the NSs gene sequence from the Phlebovirus S segment. Furthermore, using restriction enzymes, cloning and PCR technology, it is possible to replace the deleted NSs sequence with another sequence, for example a sequence encoding the Phlebovirus Gc and Gn glycoproteins. Further information regarding such techniques may be found in “Molecular Cloning: A Laboratory Manual” by Sambrook and Russell (Pub: CSHL Press).

One of skill in this field will appreciate that template nucleic acid for use in generating the modified viruses described herein may take the form of a vector comprising one or more Phlebovirus genome sequences. For example, the template nucleic acid may comprise sequences encoding the L, M and/or S genome segments.

The invention further relates to methods for producing modified virus according to this invention, said method comprising the steps of:
(a) transfecting a cell with one or more vectors encoding a modified Bunyaviridae genome segment and/or protein and one or more vectors encoding wild type Bunyaviridae genome segments and/or proteins;
(b) maintaining the transfected cell under conditions suitable to promote or induce production of virus; and
(c) rescuing virus;
wherein the rescued virus is a modified virus according to the first aspect of this invention (and or any of the embodiments thereof).

By way of example, a cell may be transfected with a vector encoding, or capable of expressing, the modified genome segments described herein. For example, the vector to be transfected may comprise (and express) a nucleic acid sequence providing a modified S segment (mS) from which the sequence encoding the NSs protein has been deleted and replaced with, for example, a sequence encoding the Gn/Gc proteins from M segment. Additionally, the cell may be transfected with one or more vectors encoding, or capable of expressing, genomic S, M and/or L segments. It should also be understood that vectors encoding, or capable of expressing Bunyaviridae N and/or L proteins, may also be used.

In one embodiment the following vectors may be used in the method for producing modified virus according to this invention:
(i) pTM1-N; For expression of RVFV N protein under T7 promoter
(ii) pTM1-L: For expression of RVFV L protein under T7 promoter
(iii) pTVT7R-GS: For expression of RVFV viral genomic S segment.
(iv) pTVT7R-GM: For expression of RVFV viral genomic M segment.
(v) pTVT7R-GL: For expression of RVFV viral genomic L segment.

The methods provided by this aspect of the invention may be further supplemented with the use of lipofectamine 2000 (invitrogen).

The term cell, as used in the third aspect of this invention (above) may encompass eukaryotic or prokaryotic cells, such as, for example, plant, insect, mammalian, fungal and/or bacterial cells. For example, suitable cells to be transfected with the vectors described herein may include mammalian cells such as Vero cells, BHK-21 or BSR-T7/5 cells.

Maintaining transfected cells under conditions suitable to promote or induce production of virus may include, for example, cells being left for approximately 5-7 days or until a cytopathic effect may be observed. To rescue virus, the media in which the cells have been maintained may be collected—the collected media containing rescued (modified) virus.

In a fourth aspect, this invention provides a vector, preferably an expression vector, comprising a nucleic acid sequence encoding the mS genome segments described herein. Expression vectors suitable for use in this aspect of the invention may further comprise one or more promoter sequences capable of directing expression in prokaryotic or eukaryotic cells such as, for example, mammalian, fungal, bacterial, plant and/or insect cells.

A vector provided by this invention may be circular or linear, single stranded or double stranded and can include DNA, RNA or a combination or modification thereof. Furthermore, vectors of this invention may be, for example, plasmids, cosmids or viral vectors (for example retroviral or bacteriophage vectors). Vectors provided by this invention may further comprise selection or marker elements, for example antibiotic resistance genes and/or optically detectable tags. A large number of suitable vectors are known and further information may be obtained from Pouwels et al. Cloning Vectors: a Laboratory Manual (1985 and supplements), Elsevier, N.Y.; and Rodriquez, et al. (ads.) Vectors: a Survey of Molecular Cloning Vectors and their Uses, Buttersworth, Boston, Mass. (1988)—both of which are incorporated herein by reference.

In a fifth aspect, the present invention provides host cells transfected with a vector as described herein. Eukaryotic or prokaryotic cells, such as, for example, plant, insect, mammalian, fungal and/or bacterial cells, may be transfected with one or more of the vectors described herein. By way of example, suitable cells to be transfected with the vectors described herein may include mammalian cells such as Vero cells, BHK-21 cells or BSR-T7/5 cells.

One of skill in this field will be familiar with the techniques used to introduce heterologous or foreign nucleic acid sequences, such as expression vectors, into cells and these may include, for example, heat-shock treatment, use of one or more chemicals (such as calcium phosphate) to induce transformation/transfection, the use of viral carriers, microinjection and/or techniques such as electroporation. Further information regarding transformation/transfection techniques may be found in Current Protocols in Molecular Biology, Ausuble, F. M., ea., John Wiley & Sons, N.Y. (1989) which is incorporated herein by reference.

In one embodiment, an expression vector comprising a nucleic acid sequence encoding a mS genome segment according to this invention may be used in a process for generating a Bunyavirus family member, for example, a modified Phlebovirus.

In one embodiment, the modified viruses described herein may be admixed with another component, such as, for example, another polypeptide and/or an adjuvant, diluent or excipient. Vaccines or vaccine compositions provided by this invention may contain fungal, viral and/or bacterial antigens used to control other diseases. For example, the vaccine or vaccine composition may be included within a multivalent vaccine which includes antigens against other diseases.

In a still further aspect, the present invention provides an ungulate or ruminant population treated or immunised with a vaccine or composition described herein. In one embodiment, the ruminant population is an ovine population.

The present invention also relates to methods of producing immune serum and/or antibodies. The methods may comprise the step of administering a modified Bunyavirus (for example a modified Phlebovirus) to test animal and, after a suitable period of time, obtaining serum. One of skill will appreciate that serum (i.e. polyclonal serum) from animals administered a modified virus according to this invention may comprise immunoglobulin (IgG, IgM, IgE, IgD, IgA and/or other isotyopes variants—depending on the site of administration (systemic/mucosal etc) and/or the animal being immunised) having affinity for or specific to, antigens of the modified virus. Since the modified viruses provided by this invention have been shown to be attenuated, the methods provided by this aspect of the invention have advantages over prior art methods of producing immune sera where modified viruses may have caused significant illness in animals, posed a serious health risk to human handlers and could easily have reverted to virulence through reassortment with other modified/wild-type strains.

The present invention further provides a vaccine for use in preventing or controlling disease in animal or mammalian hosts such as, for example, human, bovine or ovine hosts, wherein said diseases are caused or contributed to, by members of the Bunyaviridae family—for example members of the Phlebovirus genus, including RVFV.

The invention further provides a method for immunising subjects, for example animals (in particular mammals, including humans) against a Bunyavirus (for example a Phlebovirus) infection, said method comprising the step of administering to the subject a vaccine of the invention.

### DETAILED DESCRIPTION

The present invention will now be described in detail with reference to the following figures which show:

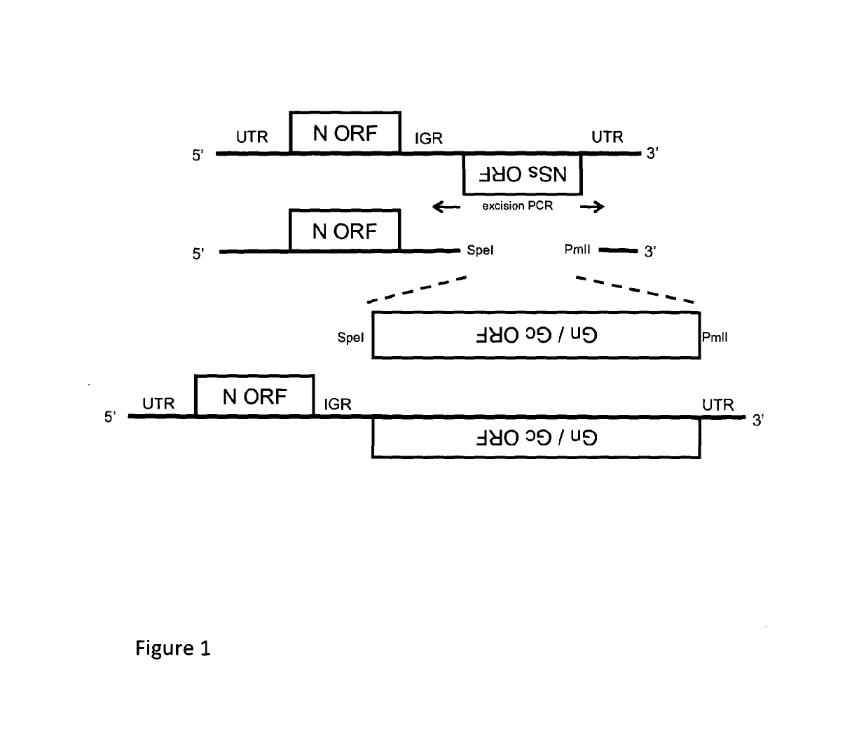

FIG. 1: Construction of the chimeric S-M genomic segment of RVFV strain MP12. The coding strategy of the S segment is shown at the top in the viral-complementary sense. Excision PCR mutagenesis was used to remove the NSs ORF and to introduce unique PmlI and SpeI restriction enzyme sites. The coding sequence for Gn and Gc was amplified from pTVT7-GM by PCR using primers that introduced PmlI and SpeI restriction enzyme sites, and allowed insertion of the Gn/Gc coding sequence into the modified S segment. UTR, untranslated region; IGR, intergenic region.

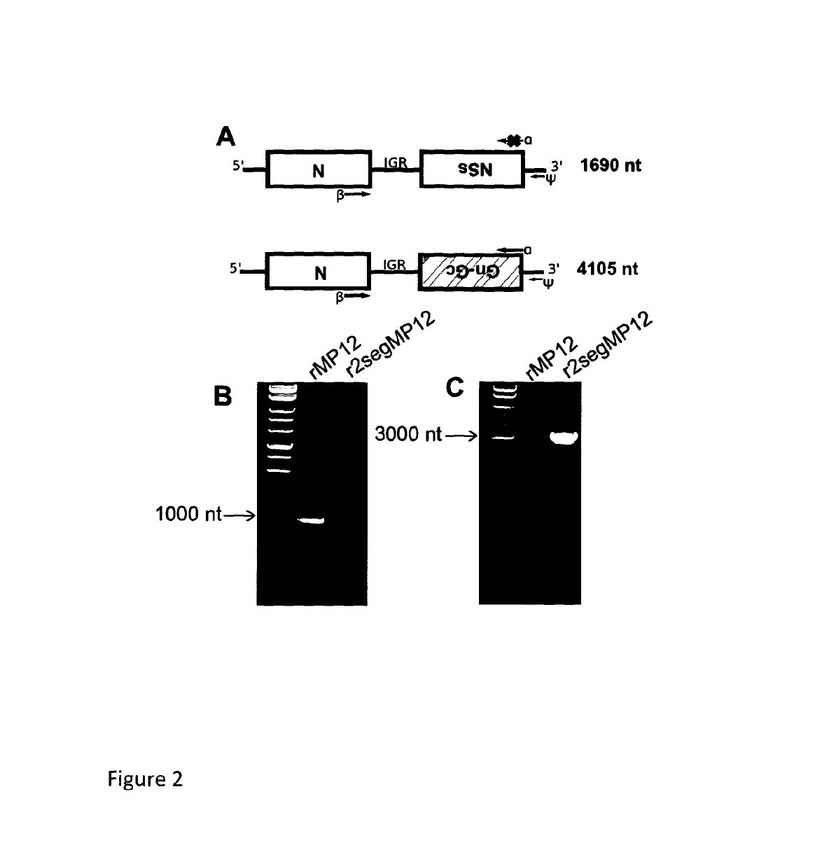

FIG. 2: Analysis of S segment RNA from three segment (MP12) or two segment (r2segMP12) recombinant viruses. (A) Schematic depiction of the parental S segment and the chimeric S-M segment, and the sites at which oligonucleotides (α, β, ψ) anneal. (B) Reverse transcription-PCR analysis was used to confirm that the two-segmented virus had an S segment that encoded the viral glycoproteins. BHK cells were infected with MP12 or the two segmented r2segMP12 viruses at a m.o.i of 1 p.f.u. per cell. Total cellular RNA was extracted from the infected cells 24 h.p.i and S segment RT-PCR was performed with the pairs of primers as indicated. No product was obtained using primer pairs α and β with MP12 S RNA as the template, as no glycoprotein encoding sequences were present for primer a to anneal. All other amplified product were the correct sizes.

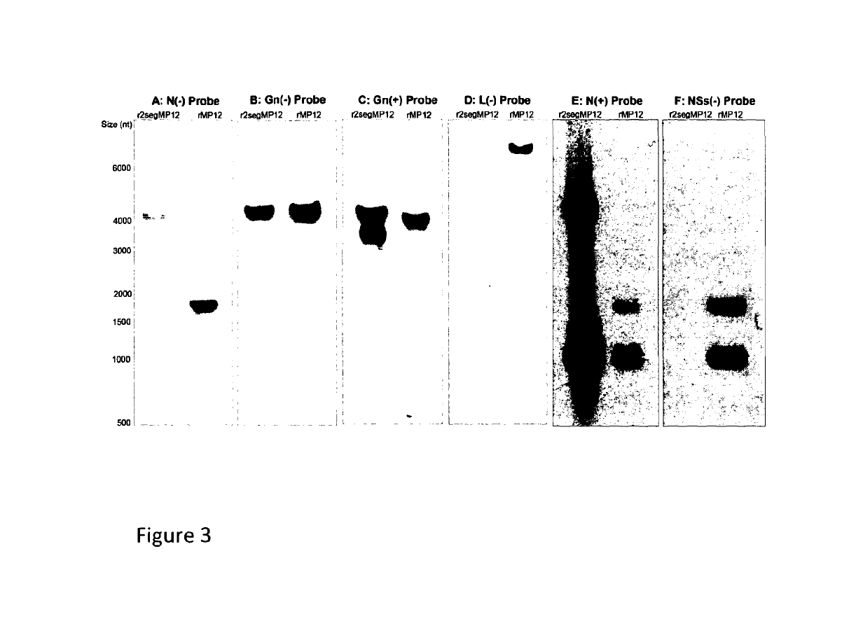

FIG. 3: Northern blot analysis of viral RNAs in infected BHK-21. Cells were infected at 1 pfu/cell with either 3-segment MP12 or 2 segment r2segMP12 virus as indicated, and total infected cell RNA was extracted at 24 hr post infection. Northern blotting was performed using DIG-labelled probes complementary to N, NSs, Gn, & L coding sequences in the viral genomic RNA (−) (A, B, D, & F) and Gn & N coding sequences in the viral anti-genomic RNA(+) (C & E). The sizes of the RNA species are indicated on the left and the sizes of the detected products were as expected,

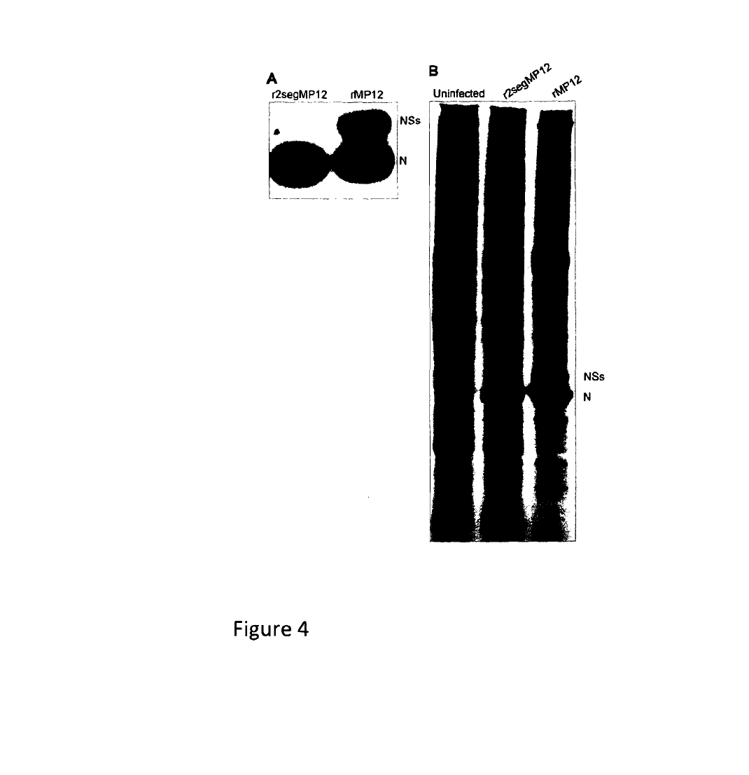

FIG. 4: Characterisation of protein expression from the recombinant two-segmented virus. (A) Western blot analysis of BHK-21 cells infected with rMP12 or r2segMP12 at a m.o.i. of 1. Cell extracts were harvested 48 h p.i and loaded into 4-12% NuPage gel (Invitrogen) and the blot was probed with anti-N and anti-NSs antibodies. No NSs band was detected in r2segMP12-infected cells. (B) Inhibition of host cell protein synthesis by rMP12 or the recombinant r2segMP12 virus. BHK-21 cells were infected at a m.o.i of 5, and harvested at 24 h p.i. The cells were labelled with 50 μCi [³⁵S]methionine for 2.5 h and cell lysates analysed by SDS-PAGE. The positions of the viral proteins N and NSs are indicated on the right.

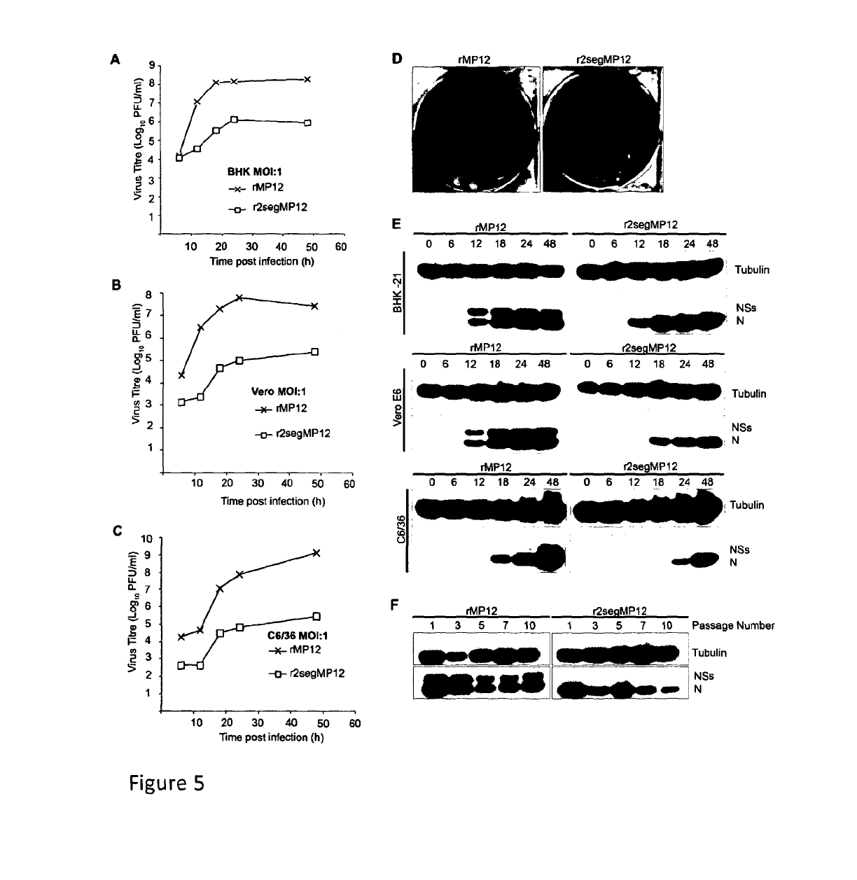

FIG. 5: Growth kinetics, viral protein synthesis, plaque phenotype and serial passage of rMP12 and the recombinant r2segMP12 viruses. Viral growth curves in (A) BHK-21, (B) Vero E6 and (C) C6/36 cells. Cells were infected with either rMP12 or r2segMP12 at a m.o.i of 1. Viruses were harvested at the time points indicated and titrated by plaque assay. (D) Comparison of plaque sizes on BHK-21 cells. Cell monolayers were fixed 96 h p.i. with 4% paraformaldehyde and stained with Giemsa solution. (E) Western blot of S segment encoded proteins from rMP12 and r2segMP12 in infected cells. In tandem with the viral titres, infected cell extracts were harvested at the time points indicated, loaded into 4-12% NuPage gel (Invitrogen) and blots were probed with anti-N, anti-NSs and anti-tubulin antibodies. (F) Serial passaging of rMP12 and r2segMP12 in BHK-21 cells. Western blots probed with anti-N, anti-NSs and anti-tubulin antibodies.

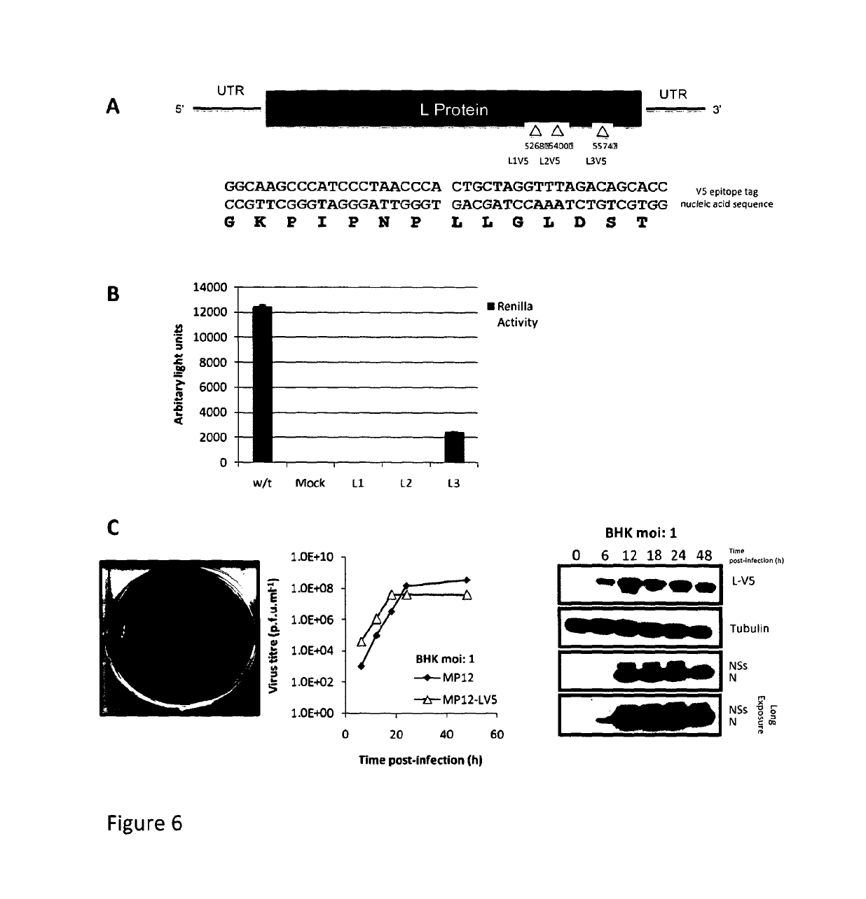

FIG. 6: Construction of a recombinant RVFV virus expressing V5 epitope-tagged L protein. (A) The 14 amino acid V5 epitope tag was inserted into the coding region of the L protein by PCR mutagenesis with both pTM1-RVFV L and pTVT7-GL DNA as template. Insertion sites are indicated by triangles at the indicated nucleotide positions and were designated L1V5-L3V5. (B) Activity of V5-tagged L proteins in a minireplicon assay. BSR-T7 cells were transfected with pTM1-RVFV N, pTVT7-Gs:hRen (encoding an MP12 S segment in which the coding sequence for the non-structural protein NSs has been replaced with that of humanised Renilla luciferase (hRen)) and either parental pTM1-RVFV L or V5-tagged L mutants. Renilla luciferase activity in cell lysates was measured at 24 h post-transfection and is shown in arbitrary light units. (C) Generation of a LV5-tagged RVFV. Construct pTVT7-GL3V5 was used in place of parental RVFV L plasmid and a recombinant virus expressing the V5 tag recovered by reverse genetics. Plaque phenotype, growth kinetics and viral protein synthesis of the recombinant virus rMP12L3V5 are shown compared to MP12 virus.

### MATERIALS AND METHODS

### Cells and Viruses

Vero cells were grown in Dulbecco's modified minimal essential medium (DMEM) supplemented with 10% fetal bovine serum (FBS). BHK-21 cells were grown in Glasgow modified MEM (GMEM) supplemented with 10% heat-inactivated FBS, and BSR-T7/5 cells, which stably express T7 RNA polymerase (Buchholz et al., 1999) were grown in GMEM supplemented with 10% FBS and 1 mg/ml G418. All mammalian cell lines were grown at 37° C. with 5% CO₂ unless otherwise stated. The Aedes albopictus derived cell line C6/36 was maintained in Leibovitz's L-15 medium supplemented with 10% FCS and 8% TPB. These cells were incubated at 28° C. in the absence of CO₂. Recombinant Rift Valley fever viruses created by reverse genetics were grown under BSL3 conditions by infecting BHK-21 cells at a multiplicity of infection (MOI) of 0.01 pfu/cell and harvesting the culture medium after incubation for 72 h. For serial blind passage of viruses at low multiplicity, infected BHK-21 cells were grown at 33° C. in medium supplemented with 2% NCS until marked cytopathic effect (cpe) was observed; the supernatant was clarified and an aliquot used to inoculate fresh BHK-21 cell monolayers.

### Plasmids

Plasmids which contain full-length cDNA copies of the RVFV MP12 genome segments or which express RVFV MP12 proteins have been described previously (Billecocq et al., 2008). To delete the NSs gene in the S segment, plasmid pTVT7-GS was used as template in an excision PCR reaction (Shi and Elliott, 2002), using outward-facing oligonucleotides 5′-acaggaaagtggtacctgatacacgtgataagcactag-3′ and 5′-gaggaggagaggtaccatgatggactagttgaggttgattag-3′. The PCR product generated lacked nt 19 to 819 of the RVFV S segment, and possessed at its 3′ end SpeI and KpnI restriction sites and at its 5′ end KpnI and PmII sites. The PCR product was digested with KpnI and religated to form plasmid pTVT7-GSΔNSs-KpnI. Digestion of pTVT7-GSΔNSs-KpnI with PmlI and SpeI allowed directional insertion of foreign genes into the S segment in an ambisense orientation. The coding sequences for enhanced GFP, humanized Renilla luciferase, and RVFV Gn-Gc sequences which were amplified by PCR from pEGFP-N1 (Clontech), phRL-CMV (Promega) or pTVT7-GM (Billecocq et al., 2008) respectively. The sequences were then verified to ensure no mutations had occurred during the cloning process. All sequences of the specific oligonucleotides used for the different constructs are available on request.

To insert the V5 epitope tag into the L protein or the genomic L segment, the plasmids pTVT7-GL and pTM1-L were used as templates in PCR mutagenesis reactions (Shi and Elliott, 2009), using outward facing oligonucleotides with an additional 5′ phosphate group. The PCR products generated contained one half of the V5 tag at their 5′ ends and the other at their 3′ ends. The resulting PCR products were ligated overnight with T4-ligase (Promega). The sequences were then verified to ensure no mutations had occurred during the cloning process. All sequences of the specific oligonucleotides used for the different constructs are available on request.

Generation of Recombinant Viruses from cDNA by Reverse Genetics

The reverse genetics protocol essentially followed that described by Lowen et al (Lowen et al., 2004). BSR-T7/5 cells (7×10⁵ cells in a T-25 flask) were transfected with the expression plasmids pTM1-L (0.5 μg) and pTM1-N (0.5 μg) together with 1 μg each of the transcription plasmids pTVT7-GL, pTVT7-GM and pTVT7-GS (to generate RVFV strain MP12) or pTVT7-GL and one of the modified S segment cDNA constructs, using 2 μl Lipofectamine 2000 (Invitrogen) per μg of plasmid DNA.

In the case of the V5 epitope-tagged L protein-containing virus, BSR-T7/5 cells (7×10⁵ cells in a T-25 flask) were transfected with the expression plasmids pTM1-L (0.5 μg) and pTM1-N (0.5 μg) together with 1 μg each of the transcription plasmids pTVT7-GL3V5, pTVT7-GM and pTVT7-GS, to generate RVFV strain MP12LV5. After 5 to 7 days, when extensive cytopathic effect was observed, the culture media were collected and stored at −80° C.

### Virus Titration by Plaque Assay

BHK cells were infected with serial dilutions of virus and incubated under an overlay consisting of DMEM, 2% FCS and 1% agarose at 37° C. for 7 days. Cell monolayers were fixed with 4% formaldehye, and plaques were visualized by staining with Giemsa.

### Virus Growth Curves

BHK-21 cells in T-25 flasks were infected at a MOI of 1.0 pfu/cell with the different viruses. One hour post-infection, the inoculum was removed and cells were washed with phosphate-buffered saline to remove unattached viruses. At the time point indicated, viruses in the supernatant fluid were titrated by plaque assay on BHK-21 cells.

### Metabolic Labeling of Viral Proteins

BHK-21 cells in T-25 flasks were infected at a MOI of 1.0 pfu/cell with the different viruses. Twenty-four hours post-infection, cells were labeled with 50 μCi per flask of [³⁵S] methionine for 2.5 h. Cell lysates were prepared and analyzed by sodium dodecyl sulfate (SDS)-polyacrylamide gel electrophoresis (PAGE) as described previously (Watret et al., 1985).

### Western Blotting

BHK-21 cells in T-25 flasks were infected at a MOI of 1.0 pfu/cell with the different viruses. Twenty-four hour post-infection cell lysates were prepared by the addition of 1 ml cell lysis buffer (100 mM Tris; 4% (wt/vol) SDS; 20% (wt/vol) glycerol; 200 mM DTT, 0.2% (wt/vol) bromophenol blue and 25 U Benzonase (Novagen)). Equal amounts of cell extract were separated by SDS-4-12% PAGE (Invitrogen) and transferred to a Hybond-C Extra membrane (Amersham), followed by 2 h incubation in saturation buffer (phosphate-buffered saline containing 5% dry milk and 0.1% Tween 20). The membrane was incubated overnight in saturation buffer containing an anti-N antibody at a dilution of 1:20,000, anti-NSs antibody at a dilution of 1:20,000, anti-tubulin antibody at a dilution of 1:5,000 (Sigma) or anti-V5 antibody (Serotec) at a dilution of 1:1000, followed by incubation with horse-radish peroxidase (HRP)-labeled anti-rabbit antibody (Cell Signaling Technology) diluted 1:1000 in saturation buffer for 1 h. After three washes in phosphate-buffered saline-0.1% Tween 20, the membrane was rinsed in phosphate-buffered saline, and detection was performed using SuperSignal WestPico chemiluminescent substrate (Pierce).

### Analysis of Viral RNA

Analysis of RNA by Northern blot was as described previously (Lowen et al., 2005). Briefly, BHK-21 cells in T-75 flasks were infected at MOI of 1.0 pfu/cell with the different viruses. Forty-eight hours post infection total cellular RNA was extracted using TRIzol reagent (Invitrogen), and 4 μg of RNA was electrophoresed through a 1.2% agarose gel. The RNA was then transferred to a positively charged nylon membrane (Roche), and hybridized with digoxigenin-labelled segment- and strand-specific RNA probes (150 ng of each probe), followed by detection with the DIG Northern starter kit (Roche).

For reverse transcription-PCR analysis, 1 μg of total cellular RNA was mixed with 100 pmole of a segment specific oligonucleotide, in buffer containing 0.5 mM each dNTP, 40 U rRNasin (Promega) and 200 U M-MLV reverse transcriptase, and incubated at 42° C. for 3 hours. The resulting cDNA was the used in PCR reactions with the primer sets ψ (5′-acacaaagaccccctagtgcttatc-3′) and β (5′-gaatagcaactttataagccatg-3′) and α (5′-gtatgagctcactgaagactgcaac-3′) and β. The first pair of primers determined the size of the inserted coding sequence, and the second confirmed the insertion of the Gn/Gc coding sequence (FIG. 2). The amplified DNA products were characterized by agarose gel electrophoresis and UV illumination.

### Results

### Rescue of Recombinant RVF Viruses.

Previous studies have shown that the RVFV NSs protein, that is encoded in an ambisense strategy in the genomic S RNA segment (Giorgi et al., 1991), is not essential for growth in tissue culture or in animals, and acts a major virulence factor (Vialat et al., 2000, Bouloy et al., 2001, Billecocq et al., 2004, Ikegami et al., 2006). The NSm coding region at the N-terminus of the glycoprotein precursor encoded on the viral M segment is similarly not essential for viral replication, and Rift Valley fever viruses lacking both the NSs and NSm genes were highly attenuated in mammalian cell culture systems and small animal models (Won et al., 2006, Bird et al., 2007b, Gerrard et al., 2007b). Such recombinant viruses have potential as live attenuated vaccines (Bird et al., 2008). However, the possibility exists that reverse to virulence could occur via genome segment reassortment with a wild type strain in the field situation (Sall et al., 1999, MacLachlan and Hirsch, 2004). In order to reduce this possibility, as well as creating a recombinant virus with a clearly distinguishable genome structure, we investigated whether we could express the viral glycoproteins in an ambisense orientation in a modified S segment in place of the NSs protein. This would obviate the need for the M genome segment, and convert RVFV into a two-segmented virus. To this end, we inserted the Gn/Gc coding sequence in place of the NSs ORF (FIG. 1), and using reverse genetics we attempted to recover infectious virus. Recovery of RVFV using T7 promoter-based plasmids into T7 polymerase expressing cells has been reported (Ikegami, Won et al. 2006; Bird, Albarino et al. 2007; Billecocq, Gauliard et al. 2008; Habjan, Penski et al. 2008, using essentially the procedures first developed for Bunyamwera virus (Lowen, Noonan et al. 2004. In this system “transcription” plasmids generate full-length RNAs corresponding to antigenome-sense RNAs and “support” plasmids express viral proteins. Therefore, BSR-T7/5 cells were transfected with transcription plasmids (based on pTVT7) containing parental MP12 L cDNA and the chimeric S segment, along with support plasmids (based on pTM1) encoding the viral N and L proteins. As control, the three parental MP12 cDNA transcription plasmids were used in a parallel transfection. Both transfections yielded virus at the first attempt; parental MP12 gave 2×10⁶ PFU/ml whereas as the transfection using the modified S segment gave 3×10⁵ PFU/ml. Independent isolates were obtained by plaque purification on BHK-21 cells, and after amplification in BHK-21 cells, the resulting viral stocks were characterised. The virus obtained using the modified S segment was designated r2segMP12.

### Confirmation of a Two Segmented RVF Virus Lacking NSs and NSm

The S segment RNA of each recovered virus was analyzed by reverse transcription-PCR using specific primers as shown in FIG. 2. Reverse transcription was primed with an oligonucleotide specific for the 3′ end of the virion-sense S segment, and the cDNA used in PCR reactions with primers designed to anneal to the glycoprotein ORF (A), the 3′ end of N ORF (B) or the S segment 3′UTR (C). A PCR product of 1041 nt was produced from MP12 derived cDNA when using primers B and C that corresponds to the viral 3′UTR, NSs ORF and intergenic region. However, as expected when primer set A and B was used, no PCR product was seen. When the r2segMP12-derived cDNA was used with primer set A and B, a product of 2983 nt was obtained, indictaing that the S RNA segment indeed contained sequences encoding the viral glycoproteins. When primer set B and C was used, a product of 3446 nt was obtained, corresponding to the predicted size of the chimeric segment. Nuceotide sequence analysis of the amplified DNA products confirmed that the recombinant virus S segments had sequences corresponding to the palsmids used for their rescue (data not shown).

Analysis of Viral RNA from Infected Cells.

The genome of r2segM12 would comprise an L segment of 6404 nt, and a chimeric S segment of 4105 nt, whereas MP12 virus has genome segment lengths of 6404 nt (L), 3885 nt (M) and 1690 nt (S). Northern blot analysis of total RNA from infected BHK-21 cells using segment and strand-specific probes provided further evidence that the recombinant MP12 and r2segMP12 viruses had the predicted genome structures. Sizes of RNA species detected by the different probes were estimated by reference to an RNA size marker ladder on the same gel that was stained with ethidium bromide. The S-probe (that hybrised to N ORF sequences) detected an RNA in MP12 virus-infected cells of approximately 1700 nt, the size expected of the native S genome segment.

However, the same S− probe detected an RNA species of approximately 4100 nt in r2segMP12 infected cells, as expected for the chimeric S segment. The L− probe detected L segment RNA with the expected size in both the MP12 and r2segMP12 infected cells. Two M segment derived probes were used, one (M−) to detect virion-sense RNA and the other (M+) to detect virion-complementary RNA. The M− probe detected an RNA species of the expected size of the M segment in MP12-infected cells, and an equivalent sized RNA to that detected by the S− probe in the r2segMP12 intracellular RNA. When the M+ probe was used, a single band was observed in MP12 infected cells, corresponding to M segment antigenome and mRNA that would comigrate on the gel. However, two RNAs were detected with this probe in r2segMP12 infected cells, the larger corresponding the full-length chimeric S-M segment, and a smaller band corresponding to the subgenomic mRNA encoding the Gn-Gc precursor. As the glycoprotein coding sequence is in the ambisense orientation in the chimeric S segment its messenger RNA would be detected by the M+ probe.

### Viral Protein Analysis.

Western blot analysis was performed on extracts of cells infected with MP12 and r2segMP12 viruses using monospecific antibodies raised against the RVFV N and NSs proteins produced in E. coli. As seen in FIG. 4A, whereas N protein was clearly detected in lysates from cells infected with either virus, NSs was only detected in MP12 virus infected cells. Metabolic labeling of infected cells with 35S-methionine produced a similar pattern. Compared to mock infected cells, a band of the appropriate size (approx 25 kDa) was seen in both MP12 and r2segMP12 virus infected cells, but a slightly slower migrating band corresponding to NSs (approx 28 kDa) was only seen in MP12 virus infected cells. Also noteworthy is that there little shut-off of host protein synthesis in r2segMP12-infected cells compared to MP12 infected cells, which correlates with the known effects of NSs in inhibiting host cell transcription. Finally, immunofluorescent staining of infected cells with monospecific antibodies confirmed that whereas N protein was detected in the cytoplasm of cells infected with both viruses (FIG. 4C), NSs was only detected (in the nucleus, as expected) in cells infected with MP12.

### Growth Characteristics of Recombinant Viruses.

The growth of r2segMP12 was compared to that of MP12 virus in BHK cells at two multiplicities of infection, 0.001 and 1.0 pfu/cell (FIG. 5A). At the lower multiplicity, growth of rMP12 was retarded compared to MP12, though both viruses reached their peak titres by 48 hr p.i. The titre of the two-segment virus was about 75-fold lower than that of the parental MP12 virus (7×10⁵ pfu/ml vs. 7×10⁷ pfu/ml). Growth kinetics were similar at the higher multiplicity, and the difference in peak titre was less marked (4.5×10⁷ pfu/ml vs. 3.5×10⁸ pfu/ml). Cell extracts taken from the same cultures as above showed that the synthesis of viral N and NSs (where synthesized) as monitored by Western blot analysis with monospecific antibodies reflected the growth curves FIG. 5B).

The plaque phenotypes of the two-segmented r2segMP12 and the parental MP12 virus were examined in BHK cells (FIG. 5C). The r2segMP12 viral plaques were consistently smaller than MP12, consistent with the growth kinetics observed in the time course experiments.

### Generation of Recombinant Viruses Expressing V5-Tagged L Protein

Bunyavirus L proteins are difficult to detect in infected cells, but recently we described the generation of a recombinant BUNV expressing L protein tagged with the V5-epitope (Shi and Elliott, 2009); the epitope was inserted towards the C terminus of the protein. We sought to create the analogous insertion of the epitope into the RVFV L protein, and based on comparison of available phlebovirus L protein sequences (data not shown) we selected 3 positions to insert the V5 epitope (FIG. 6 A). The effect of inserting the 14-residue sequence on L protein function was first assessed in the RVFV minireplicon system (Gauliard et al., 2006). No minireplicon activity was observed when the epitope was inserted at positions L1 and L2, but a modest activity, approx 17% of unmodified L protein, was observed when the epitope was inserted in the most C-terminal position, L3 (FIG. 6B). Therefore, virus rescue was attempted with the modified L construct, and a recombinant virus was recovered and designated rMP12-LV5. This virus gave a smaller plaque size, and grew to slightly lower titres, than parental MP12 FIG. 6 C). Analysis of viral proteins by Western blotting revealed that the anti-V5 antibody clearly detected the tagged L protein by 6 hr pi. (FIG. 6C).

### REFERENCES

Billecocq, A., Gauliard, N., Le May, N., Elliott, R. M., Flick, R. & Bouloy, M. (2008) RNA polymerase I-mediated expression of viral RNA for the rescue of infectious virulent and avirulent Rift Valley fever viruses. Virology, 378, 377-384.
Billecocq, A., Spiegel, M., Vialat, P., Kohl, A., Weber, F., Bouloy, M. & Haller, O. (2004) NSs protein of Rift Valley fever virus blocks interferon production by inhibiting host gene transcription. Journal of Virology, 78, 9798-806.
Bird, B. H., Albarino, C. G. & Nichol, S. T. (2007a) Rift Valley fever virus lacking NSm proteins retains high virulence in vivo and may provide a model of human delayed onset neurologic disease. Virology, 362, 10-5.
Bird, B. H., Albarino, C. G., Hartman, A. L., Erickson, B. R., Ksiazek, T. G. & Nichol, S. T. (2008) Rift Valley Fever Virus Lacking the NSs and NSm Genes Is Highly Attenuated, Confers Protective Immunity from Virulent Virus Challenge, and Allows for Differential Identification of Infected and Vaccinated Animals. J. Virol., 82, 2681-2691.
Bird, B. H., Albarino, C. G. & Nichol, S. T. (2007b) Rift Valley fever virus lacking NSm proteins retains high virulence in vivo and may provide a model of human delayed onset neurologic disease. Virology, 362, 10-15.
Bouloy, M. & Flick, R. (2009) REVERSE GENETICS TECHNOLOGY FOR RIFT VALLEY FEVER VIRUS: CURRENT AND FUTURE APPLICATIONS FOR THE DEVELOPMENT OF THERAPEUTICS AND VACCINES. Antiviral research.
Bouloy, M., Janzen, C., Vialat, P., Khun, H., Pavlovic, J., Huerre, M. & Haller, O. (2001) Genetic evidence for an interferon-antagonistic function of rift valley fever virus nonstructural protein NSs. J Virol, 75, 1371-7.
Buchholz, U. J., Finke, S. & Conzelmann, K. K. (1999) Generation of bovine respiratory syncytial virus (BRSV) from cDNA: BRSV NS2 is not essential for virus replication in tissue culture, and the human RSV leader region acts as a functional BRSV genome promoter. J Virol, 73, 251-9.
Elliott, R. M. (2009) Bunyaviruses and climate change. Clin Microbiol Infect, 15, 510-7.
Gauliard, N., Billecocq, A., Flick, R. & Bouloy, M. (2006) Rift Valley fever virus noncoding regions of L, M and S segments regulate RNA synthesis. Virology, 351, 170-179.
Gerrard, S. R., Bird, B. H., Albari√±O, C. G. & Nichol, S. T. (2007a) The NSm proteins of Rift Valley fever virus are dispensable for maturation, replication and infection. Virology, 359, 459-65.
Gerrard, S. R., Bird, B. H., Albarino, C. G. & Nichol, S. T. (2007b) The NSm proteins of Rift Valley fever virus are dispensable for maturation, replication and infection. Virology, 359, 459-465.
Habjan, M., Pichlmair, A., Elliott, R. M., Overby, A. K., Glatter, T., Gstaiger, M., Superti-Furga, G., Unger, H. & Weber, F. (2009) NSs protein of rift valley fever virus induces the specific degradation of the double-stranded RNA-dependent protein kinase. J Virol, 83, 4365-75.
Ikegami, T., Narayanan, K., Won, S., Kamitani, W., Peters, C. J. & Makino, S. (2009a) Dual functions of Rift Valley fever virus NSs protein: inhibition of host mRNA transcription and post-transcriptional downregulation of protein kinase PKR. Annals of the New York Academy of Sciences, 1171 Suppl 1, E75-85.
Ikegami, T., Narayanan, K., Won, S., Kamitani, W., Peters, C. J. & Makino, S. (2009b) Rift Valley fever virus NSs protein promotes post-transcriptional downregulation of protein kinase PKR and inhibits eIF2alpha phosphorylation. PLoS pathogens, 5, e1000287.
Ikegami, T., Won, S., Peters, C. J. & Makino, S. (2006) Rescue of Infectious Rift Valley Fever Virus Entirely from cDNA, Analysis of Virus Lacking the NSs Gene, and Expression of a Foreign Gene. J. Virol., 80, 2933-2940.
Kamal, S. A. (2009) Pathological studies on postvaccinal reactions of Rift Valley fever in goats. Virol J, 6, 94.
Lowen, A. C., Boyd, A., Fazakerley, J. K. & Elliott, R. M. (2005) Attenuation of bunyavirus replication by rearrangement of viral coding and noncoding sequences. J Virol, 79, 6940-6.
Lowen, A. C., Noonan, C., Melees, A. & Elliott, R. M. (2004) Efficient bunyavirus rescue from cloned cDNA. Virology, 330, 493-500.
Maclachlan, D. & Hirsch, D. (2004) Vaccines. 1N Hirsch, D., Nj, M. & R1, W. (Eds.) Veterinary Microbiology. 2nd ed., Wiley-Blackwell.
SalI, A. A., Zanotto, P. M. D. A., Sene, O. K., Zeller, H. G., Digoutte, J. P., Thiongane, Y. & Bouloy, M. (1999) Genetic Reassortment of Rift Valley Fever Virus in Nature. J. Virol., 73, 8196-8200.
Shi, X. & Elliott, R. M. (2002) Golgi localization of Hantaan virus glycoproteins requires coexpression of G1 and G2. Virology, 300, 31-8.
Shi, X. & Elliott, R. M. (2009) Generation and analysis of recombinant Bunyamwera orthobunyaviruses expressing V5 epitope-tagged L proteins. J Gen Virol, 90, 297-306.
Shoemaker, T., Boulianne, C., Vincent, M. J., Pezzanite, L., Al-Qahtani, M. M., Al-Mazrou, Y., Khan, A. S., Rollin, P. E., Swanepoel, R., Ksiazek, T. G. & Nichol, S. T. (2002) Genetic analysis of viruses associated with emergence of Rift Valley fever in Saudi Arabia and Yemen, 2000-01. Emerg Infect Dis, 8, 1415-20.
Smithburn, K. C. (1949) Rift Valley fever; the neurotropic adaptation of the virus and the experimental use of this modified virus as a vaccine. Br J Exp Pathol, 30, 1-16.
Vialat, P., Billecocq, A., Kohl, A. & Bouloy, M. (2000) The S segment of rift valley fever phlebovirus (Bunyaviridae) carries determinants for attenuation and virulence in mice. J Virol, 74, 1538-43.
Vialat, P., Muller, R., Vu, T. H., Prehaud, C. & Bouloy, M. (1997) Mapping of the mutations present in the genome of the Rift Valley fever virus attenuated MP12 strain and their putative role in attenuation. Virus research, 52, 43-50.
W.H.O (2008) Rift Valley Fever in Sudan—update 5. Global Alert and Response.
Watret, G. E., Pringle, C. R. & Elliott, R. M. (1985) Synthesis of bunyavirus-specific proteins in a continuous cell line (XTC-2) derived from Xenopus laevis. J. Gen. Virol., 66, 473-482.
Won, S., Ikegami, T., Peters, C. J. & Makino, S. (2006) NSm and 78-Kilodalton Proteins of Rift Valley Fever Virus Are Nonessential for Viral Replication in Cell Culture. J. Virol., 80, 8274-8278.
Won, S., Ikegami, T., Peters, C. J. & Makino, S. (2007) NSm protein of Rift Valley fever virus suppresses virus-induced apoptosis. Journal of virology, 81, 13335-45.

What is claimed is:

1.-20. (canceled)

21. A modified virus of the Bunyavirus family, said modified virus comprising a bi-segmented genome.

22. The modified virus according to claim 21 wherein the virus is a member of the Hantavirus genus, the Nairovirus genus, the Tospovirus genus, the Orthobunyavirus genus, or the Phlebovirus genus.

23. The modified virus according to claim 21 wherein the virus is a member of the phlebovirus genus and is a species of Alenquer virus, Candiru virus, Chagres virus, Naples virus, Punta Toro virus, Rift Valley fever virus, Sicilian virus, or a Toscana virus.

24. The modified virus according to claim 21 wherein the modified virus is a modified Rift Valley fever virus (RVFV).

25. The modified virus according to claim 21 which comprises, consists or consists essentially of a large (L) segment and a modified small (mS) segment.

26. The modified virus according to claim 25 wherein L segment has been modified to include one or more nucleic acid insertions, deletions and/or inversions.

27. The modified virus according to claim 25 wherein L segment has been modified to include one or more nucleic acid sequences encoding amino acid, peptide, polypeptide, or protein moieties for use as tags or labels.

28. The modified virus according to claim 25 wherein the mS segment has been modified so as to disrupt the sequence encoding the NSs protein.

29. The modified virus according to claim 28 wherein all or part of the NSs sequence has been deleted in order to prevent, reduce, or ablate NSs expression.

30. The modified virus according to claim 28 wherein one or more nucleic acid sequences (including one or more single or polynucleotide sequences) has been inserted to the NSs sequence.

31. The modified virus according to claim 28 wherein the mS segment has been modified such that at least a portion or all of the NSs sequence is replaced with one or more heterologous sequence(s) or one or more sequence(s) normally encoded by another Bunyavirus family member or genome segment.

32. The modified virus according to claim 31 wherein the heterologous nucleic acid sequence encodes proteins normally encoded by the L and/or M segments of a Bunyavirus genome.

33. The modified virus according to claim 30 wherein the NSs sequence or a portion thereof has been replaced with a sequence encoding a Bunyavirus glycoprotein Gn or Gc.

34. The modified virus according to claim 30 wherein the mS segment is a chimeric segment having an ambisense arrangement encoding the Bunyavirus N protein and the Bunyavirus glycoproteins, Gn and Gc.

35. A method of raising an immune response in an ungulate or ruminant animal comprising the step of administering a modified virus according to claim 21 to an animal.

36. A vaccine or vaccine compositions comprising a modified virus according to claim 21 for raising an immune response.

37. A method for producing a modified virus according to any one of claim 21, said method comprising the steps of:
(a) transfecting a cell with one or more vectors encoding a modified Bunyaviridae genome segment and/or protein and one or more vectors encoding wild type Bunyaviridae genome segments and/or proteins;
(b) maintaining the transfected cell under conditions suitable to promote or induce production of the virus; and
(c) rescuing the virus.

38. A vector comprising a nucleic acid sequence encoding a modified mS genome segment which is a chimeric segment having an ambisense arrangement encoding a Bunyavirus N protein and Bunyavirus glycoproteins Gn and Gc.

39. A host cell transfected with a vector according to claim 38.

40. The vector of claim 38 wherein the vector is an expression vector.


SEQUENCE LISTING (6 sequences)

SEQ ID NO: 1 - DNA, 38 bp, Rift Valley fever virus
acaggaaagtggtacctgatacacgtgataagcactag

SEQ ID NO: 2 - DNA, 42 bp, Rift Valley fever virus
gaggaggagaggtaccatgatggactagttgaggttgattag

SEQ ID NO: 3 - DNA, 25 bp, Rift Valley fever virus
acacaaagaccccctagtgcttatc

SEQ ID NO: 4 - DNA, 23 bp, Rift Valley fever virus
gaatagcaactttataagccatg

SEQ ID NO: 5 - DNA, 25 bp, Rift Valley fever virus
gtatgagctcactgaagactgcaac

SEQ ID NO: 6 - DNA, 42 bp, Simian virus 5
ggcaagcccatccctaacccactgctaggtttagacagcacc